(19) United States
(12) Patent Application Publication
(10) Pub. No.: US 2021/0322397 A1
(43) Pub. Date: Oct. 21, 2021

(54) METHOD TO PREVENT AND TREAT GINGIVAL RECESSION BY CALCIUM CHANNEL BLOCKERS, ANGIOTENSIN CONVERTING ENZYME INHIBITORS, AND ANGIOTENSIN RECEPTOR BLOCKERS

(71) Applicant: Assa Weinberg, Los Angeles, CA (US)

(72) Inventor: Assa Weinberg, Los Angeles, CA (US)

(21) Appl. No.: 17/360,466

(22) Filed: Jun. 28, 2021

Related U.S. Application Data
(63) Continuation of application No. PCT/US2019/068083, filed on Dec. 20, 2019.
(60) Provisional application No. 62/786,380, filed on Dec. 29, 2018.
(60) Provisional application No. 62/786,382, filed on Dec. 29, 2018.
(60) Provisional application No. 62/786,383, filed on Dec. 29, 2018.

(51) Int. Cl. A61K 31/4422; A61K 31/401; A61K 31/403; A61K 31/4178; A61K 31/4245; A61K 31/4184; A61K 31/34; A61K 31/345; A61K 31/04; A61K 31/517; A61K 31/216; A61K 31/138; A61K 31/167; A61K 31/165; A61K 31/549; A61K 31/50; A61P 1/02

(52) U.S. Cl. CPC A61K 31/4422; A61P 1/02; A61K 31/403; A61K 31/4178; A61K 31/4245; A61K 31/4184; A61K 31/34; A61K 31/345; A61K 31/04; A61K 31/517; A61K 31/216; A61K 31/138; A61K 31/167; A61K 31/165; A61K 31/549; A61K 31/50; A61K 31/401

(57) ABSTRACT

A method is provided to prevent and to treat gingival recession by using Calcium Channel Blockers, Angiotensin-Converting Enzyme (ACE) Inhibitors, or Angiotensin Receptor Blockers (ARB), and more particularly, to a method to prevent and treat gingival recession, e.g., age related gingival recession, by using Calcium Channel Blockers, Angiotensin-Converting Enzyme Inhibitors, or Angiotensin Receptor Blockers that are not taken orally, but administered by injection into the affected tissue or administered by topical application to the tissue of the gum, so as to increase the capillary network and blood supply to the cartilage, bones, ligaments, and soft tissues.

### INCORPORATION BY REFERENCE TO RELATED APPLICATION

This application is a continuation of PCT Application No. PCT/US2019/068083, filed Dec. 20, 2019, which claims the benefit of U.S. Provisional Application No. 62/786,380, filed Dec. 29, 2018, U.S. Provisional Application No. 62/786,382, filed Dec. 29, 2018, and U.S. Provisional Application No. 62/786,383, filed Dec. 29, 2018. Each of the aforementioned applications is incorporated by reference herein in its entirety, and each is hereby expressly made a part of this specification.

### FIELD OF THE INVENTION

A method is provided to prevent and to treat gingival recession by using Calcium Channel Blockers, Angiotensin-Converting Enzyme (ACE) Inhibitors, or Angiotensin Receptor Blockers (ARB), and more particularly, to a method to prevent and treat gingival recession, e.g., age related gingival recession, by using Calcium Channel Blockers, Angiotensin-Converting Enzyme Inhibitors, or Angiotensin Receptor Blockers that are not taken orally, but administered by injection into the affected tissue or administered by topical application to the tissue of the gum, so as to increase the capillary network and blood supply to the cartilage, bones, ligaments, and soft tissues.

### DETAILED DESCRIPTION

The gum is a pink, soft membrane that covers the anterior surface of the semi-circular bones that form the oral cavity. The maxilla and the mandible are alveolar bone structures that harbor the tooth sockets. The gum (gingiva) hugs the teeth neck and covers the alveolar sockets and the alveolar portion of the jaw. The biological structure of the gum consists of connective tissue cells and a rich network of capillaries that seal the tooth pocket from the naturally aggressive chemical and mechanical environment of the oral cavity.

Gingival recession is a painless, age related, natural process that shrinks the height of the gingival membrane. Clinically significant gingival recession is defined when the gingival margin is apical to the enamel cementum junction. A 3 mm recession is a required criterion. If 2 mm was used as a criterion that would have included 80% of the world adult population, and 90% of those above the age of 55.

The importance of this seemingly benign but universal process did not attract the attention of poets or scientists. For centuries the apparent simplicity of the gum structure belied its colossal importance to human health. No other age related biological process is responsible for affecting such a large proportion of the world's population than gingival recession. Gingival recession is the mother of all periodontal diseases worldwide.

Periodontal or gum diseases are broadly classified as gingivitis and periodontitis. These conditions afflict the adult world populations more than any other human disease. Advances in research over the last 30 years have led to fundamental changes of our understanding of the scale and prevalence of gingival recession and its impact in the world.

Humans have two sets of teeth. The first consists of 20 teeth that appear between 6 months to 2 years of age, which shed between 6 years to twelve years of age. The second set appears progressively and consists of two sets of 16 teeth, one set is embedded in the maxillary bone, the other set in the mandibular bone. Each half of the dental set contains two incisors, one canine, two premolars, and three molars. The visible portion of the tooth is the crown. The invisible portion is the root. The crown and the root meet at the neck. The hard portion of the teeth consists of the enamel, dentin and the cementum. The soft tissue of the teeth consists of the pulp, periodontal membrane and the gingiva.

The tooth crown is covered with enamel. Enamel is the hardest material in the human body. The enamel ultrastructure consists of 99% of hydroxyapatite crystals embedded in 1% organic matrix. Enamel is synthesized by the ameloblasts cells. These cells which are present during tooth development, deposit tooth enamel, the hard outer layer of the tooth which is the surface of the crown. Harder and denser than bone, because of higher content in molecular calcium, the dentin composes the core mass of a tooth. Dentin is synthesized by the odontoblasts cells, that form the outer layer of the pulp. Dentin tubules project out of the odontoblast layer from which the dentin is laid into the tooth core. The center of the tooth is occupied by the pulp system. It consists of a canal which starts at the apical foramen and transverses along the root toward the tooth crown and ends in the tooth chamber.

The tooth is firmly held in place by the cementum. The cementum is produced by the cementocytes. Cementocytes generate a bony ultrastructure which contains lacunas. These are inter connected by canaliculi from which the cementum material is laid. The cementum hugs the root's dentin from the apex to the tooth neck. Sharpey's fibers are thick collagen bundles that originate from the alveolar bone that traverse through the periodontal membrane and land into the cementum. They serve as biological tooth anchor. The pulp canal houses the vascular system that supply by arteriolar network, nutrients and oxygen for anabolic activity of the ameloblasts, odonoblasts, and cementocytes. The tooth veins remove the waste and metabolic debris from the pulp chamber travel along the pulp canal, exit through the apical foramen to join the oral cavity venous system.

Over the last three decades multiple epidemiological surveys that attempted to establish the prevalence of dental disease revealed the staggering incidence of gingival recession. The 1960-1962 First National Health and Nutrition Examination Survey (NHANS) of adults age 18 to 64 found 89% of men and 79% of women had at least one site of gingival recession. In the third (NHANS) 1988-94 50% of adults were found to have recession in at least three or four teeth. Using data from the 2009-2010 National Health Nutrition Examination Survey, the CDC Periodontal Disease Surveillance Workshop estimated the prevalence of gingival recession and periodontitis in the adult US population. Attachment loss and probing depth were measured at 6 sites per tooth for all teeth, except for third molar. 64.8 million adults above the age of 65 or 23% of the US adult population had one or more teeth with attachment loss of 3 mm, and probing depth of more than 4 mm. 40% of young adults and up to 88% of older adults have at least one site with 1 mm recession. Worldwide 15 to 20 percent of adults age 35 to 44 have severe periodontal diseases. 50 percent of children have gingivitis by the age of four and 100 percent at puberty.

The prevalence of dental caries was assessed by the 1999-2002 National Health and Nutrition Examination Study. The percent of children and adolescents who had dental caries was 41% and 90% of adults American had caries as well.^{[12]}

Gingival recession is directly implicated in 5 disease processes that initiate by the loss of gum protection: dentin hyper sensitivity; root caries and abrasion; cervical wear; tooth mobility; and dental periodontitis. Gingival recession occurs most commonly at the site of the first and second molars of the maxillary and mandibular bone. These are clinically the most encountered sites of dental plaques. Dental plaque is composed of bacterial biofilm that colonize the tooth surface. Bacteria such as S. mutans, use the dietary sucrose to produce polysaccharide polymers known as glycan to form a hardened protective membrane that glue to the tooth surface. This membrane and its tooth attachment are immune and cannot be removed by the mechanical forces of tooth brushing.

Dental plaque bacteria, in particular Streptococcus mutans are acidogenic. They secrete very low pH bacterial acids that slowly liquefy the hardened dental surface matrix, and releases the hydroxyl appetite enamel crystals. This release demineralizes the teeth and causes local tooth decay. Dental plaque bacterial invasion of the gingival membrane is the main pathological cause of gingivitis and periodontitis. Gingivitis and periodontitis are two different diseases. The bacterial involvement and the physiopathology are distinctly different, but it is the bone involvement seen only in periodontitis that separate the two.

Tooth caries are localized destruction of a tooth's hard surface by dental plaque. They occur where gingival recession has formed at the coronal surface above, or the root surface. Coronal caries are found in toddlers and the prevalence rapidly increases from childhood to age mid 40, then coronal caries incidence plateaus thereafter. Root caries differ. They continue to increase dramatically with advanced age. Gingivitis is the most common periodontal disease of childhood worldwide. This is when the gums are the only structure affected. Gingivitis is a reversible condition. Most humans have clinical signs of gingival inflammation in one or more sites of their mouth. Periodontitis is clinically identified by the use of a Williams probe. Probing-produced bleeding is the gold standard. It is associated with bone loss. Data from NHNES 3 found that the incidence of periodontitis increases from 8 percent at age 30 to more than 35 percent at age 60. Periodontal disease is the main cause of dental loss. 25 percent of the US adult population above the age of 60 have complete dental loss. Half of them from dental caries the other half from periodontal diseases.

Four biological factors are now recognized to impact the prevalence of gingival recession worldwide.

Age: both the prevalence and the severity of gingival recession are age related. This was invariably documented by all national surveys since the first 1960-1962 study. In the 1985-1986 national survey of employed adults. A 2 mm recession was present in 70% of in those aged 35 to 44. The same 2 mm recession was found in 90% of those aged 55 to 64. A 4 mm recession was already present in 13.8% of those 25 to 34 years of age, and 56.6 in those 55 to 64 years old.

Gender: both the prevalence and severity of gingival recession are more common in men than women. This was a consistent finding in all the NHANES since 1960. The reason for the gender differences is attributed to poorer oral hygiene, poorer attitude toward oral health, and less dental office visits.

Genetics: certain genes were found to be associated with increase in loss of gingival height. A specific genotype of the polymorphic IL1 gene was identified as a contributory cause to gingival recession and periodontitis among some patient's groups. IL1 is a key regulator of host response to bacterial infection. An identical twin study among 169 twin pairs concluded that half of the prevalence of periodontitis, i.e. gingival recession, was attributable to hereditability.

Poor oral hygiene: A longitudinal study among Norwegian professionals and students, among whom oral hygiene was excellent, found no age related increase in prevalence or severity between late teen years and age 40. While in a 15 year longitudinal study of 480 Sri Lankan tea workers where none of the subjects received any dental treatment. The data reflects the natural history of gingival recession. Both oral hygiene and gingival clinical conditions were poor at all ages.

Overzealous tooth brushing: A systemic review of 17 studies, brushing force, brushing frequency, (more than one time a day) duration of brushing, frequency of changing tooth brushes, toothbrush hardness and tooth brush technics, were all found to contribute to gingival recession.

Smoking: smoking is a risk factor independent of age, gender, poor oral hygiene or other risk factors. This was identified in the 1970-1975 National Health and Nutritional Survey. The evidence continues to mount since then.

Oral contraceptives: oral contraceptives have long been considered causative for gingival recession, gingival bleeding, and other periodontal processes. Lower dosages of modern oral contraceptives not appear to negatively affect the recession or inflammation produced by dental plaques.

Diabetes mellitus: patients with type 1 and type 2 diabetes mellitus have an increased risk for periodontal disease. 15 percent of adolescents with type 1 diabetes have periodontal disease. Poor diabetic control leads to more severe gingivitis than in healthy children. Periodontal diseases in turn worsen diabetes control.

Cardiovascular diseases: the magnitude of cardiovascular risks: angina, myocardial infarction and fatal coronary heart disease, was studied among 1203 men that were followed for a median of 24 years. Coronary artery disease was compared to radiographic bone loss in the oral cavity. A double increase in coronary events was found among men less than 60 years of age with bone loss, (a 2.12 hazard ratio), but not in older individuals.

Chronic poor oral health is associated with increase in coronary and cerebrovascular disease. This effect appears to persist after adjustment for all known cardiovascular risk factors. The mechanism is not well understood. The role of vascular endothelial function is suspected.

The impact of blood supply and endothelial function on gingival dental attachment and recession was never studied or suspected.

5 predisposing risk factors related to a specific anatomy of the oral cavity were identified that impact gingival height: bone dehiscence, tooth malposition, thin gingiva, inadequate keratinized mucosa, and Frenum pull were all found to affect the incidence of gingival recession.

An Additional four factors can precipitate gingival recession: traumatic forces, oral piercing, plaque induced inflammation, and certain types of orthodontic tooth movement or sub gingival restoration.

In spite of the staggering prevalence, and the significant scientific advances in the physiopathology of periodontal diseases, only three preventive modes were found to impact the gingival recession incidence and outcomes: smoking cessation, instruction of appropriate tooth brushing technic, and periodic dental office visits which include mechanical manipulation of dental plaques. Tooth brushing, dental flossing or antibiotics cannot dislodge dental plaque attachment to teeth. The only known method capable of detaching the polysaccharides dental plaques membrane from tooth surface is instrumental manipulation by professional dentists. This data highlights the limited role of prevention and the restricted role of treatment of gingival recession.

In spite of the astonishing prevalence of gingival recession worldwide and the major health contribution that prevention will usher, no pharmacological intervention that can prevent, delay, treat or reverse the progression of gingival recession was ever published. In particular, no toothpaste containing gingival recession preventing or treating product has been discovered, developed or produced.

### SUMMARY OF THE INVENTION

The present formulations are the first such discovery, and a conceptual breakthrough. It reveals the existence of the first in a new class of pharmaceutical products that are able to prevent, delay treat or reverse the process of gingival recession.

Currently, no toothpaste anywhere in the world contains a pharmaceutical entity that is able to prevent, delay, treat or reverse the gingival recession process.

The embodiments are directed to a method for preventing gingival recession by direct administration of a pharmaceutical preparation of a vasodilator, e.g., an angiotensin receptor blocker, ACE inhibitor, or calcium channel blocker, to the gingival membrane to prevent the process and symptoms of gingival recession.

By gingival recession as used herein, it is meant any processes in the oral cavity that cause the appearance and symptoms of gingival recession.

In one method, the vasodilator, e.g., an angiotensin receptor blocker, ACE inhibitor, or calcium channel blocker may be administered directly to the gingival membrane of the oral cavity before recession is formed to prevent gingival recession. For example, in child, adolescent, men or women with normal gingival height before the recession process started.

In another embodiment, the vasodilator, e.g., an angiotensin receptor blocker, ACE inhibitor, or calcium channel blocker may be administered directly to the gingival membrane to treat and heal the process of gingival recession.

The vasodilator, e.g., an angiotensin receptor blocker, ACE inhibitor, or calcium channel blocker may be administered even after the gingival recession has dissipated and the gingival membrane has healed to prevent the recurrence of gingival recession.

Contact vasodilators, e.g., an angiotensin receptor blocker, ACE inhibitor, or calcium channel blocker, are a new class of pharmaceutical products. When applied directly to body membranes they cause an increase in capillary network, augment capillary blood supply, and enhance tissue repair in diverse body membrane.

Vasodilators, e.g., angiotensin receptor blockers, ACE inhibitors, or calcium channel blockers, were extensively studied but their ability to prevent and treat gingival recession remained unknown.

The present invention introduces the use of application of a vasodilator, e.g., an angiotensin receptor blocker, ACE inhibitor, or calcium channel blocker, to the gingival membrane space of the oral cavity. Direct contact of vasodilator, e.g., an angiotensin receptor blocker, ACE inhibitor, or calcium channel blocker, with the gingival membrane increases the capillary network and augments the blood supply to the gingival membrane and in particular, increases the capillary network and augments the blood supply to the gingival membrane territory.

The new class of contact vasodilator, e.g., an angiotensin receptor blocker, ACE inhibitor, or calcium channel blocker, may be used for the prevention and treatment of age related gingival recession or other syndromes associated with gingival recession.

No trial of a vasodilator, e.g., an angiotensin receptor blocker, ACE inhibitor, or calcium channel blocker, for the prevention and treatment of gingival recession was ever published.

### DETAILED DESCRIPTION OF THE INVENTION

The following discussion addresses a number of embodiments and applications of the present disclosure. The beneficial features of the present disclosure will be evident from the described embodiments. It is to be understood that the present disclosure is not limited to such specific applications and that numerous implementations of the present disclosure may be realized. All references to patents, patent applications, and non-patent publications mentioned in the specification are hereby incorporated by reference, in their entireties.

Neo contact-vasodilators are a new class of medication. Provided herein are methods of using neo contact-vasodilators such as calcium channel blockers (CCB), angiotensin receptor antagonists, and ACE Inhibitors, known in the treatment of hypertension and congestive heart failure, for the treatment of gingival recession.

Angiotensin Receptor Blockers, such as Valsartan, telmisartan, olmesartan, losartan, irbesartan, candesartan and azilsartan, when administered directly to the gum, are very effective drugs for the prevention and treatment gingival recession, and in particular, age-related age-related gingival recession.

ACE Inhibitors are a class of pharmaceutical drugs, that when taken orally or intravenously, dilates the arteriolar system by blocking the entry of calcium to special cells. When applied directly to body membranes, they cause an increase in capillary network, augment capillary blood supply, and enhance tissue repair in diverse body membrane. Currently, there are 20 pharmaceutical patented ACE Inhibitor drugs that use this property to treat hypertension, congestive heart failure. The clinical indication of Ace Inhibitors is therefore limited to the field of cardiovascular diseases.

Calcium Channel Blockers (CCB) are a class of pharmaceutical drugs, that when taken orally or intravenously, dilates the arteriolar system by blocking the entry of calcium to special cells. When applied directly to body membranes they cause an increase in capillary network, augment capillary blood supply, and enhance tissue repair in diverse body membrane. Currently, there are nearly 60 pharmaceutical patented Calcium Channel Blocker drugs that use this property to treat hypertension, angina pectoris, and cardiac arrhythmia. The clinical indication of Calcium Channel Blockers is therefore limited to the field of cardiovascular diseases. Calcium Channel Blockers were extensively studied but their ability to prevent and treat gingival recession remained unknown in the literature.

Contact-vasodilators, when applied in a pharmacological composition in an effective amount directly into the gingival membrane by direct application or by injection, are effective drugs for the treatment of a gingival recession.

[symbol]

Administration of vasodilators, which previously may have been used in the treatment of high blood pressure, and congestive heart failure, in a pharmacological composition, in a suitable amount, are effective drugs for the prevention of gingival recession and in particular, age-related gingival recession.

Pharmacological composition as used herein is a pharmaceutical preparation according to the invention, composed but not limited to a vasodilator and a suitable non-toxic pharmaceutical carrier.

Effective amount as used herein is an amount of pharmaceutical composition of vasodilator that is effective for treating of gingival recession. An amount of contact-vasodilator that is suitable for direct application or an injection to the gingival membrane.

A method is provided of applying a pharmaceutical preparation in an effective amount of one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), topically or by injection to treat gingival recession.

The pharmacological preparation can comprise a calcium channel blocker. The calcium channel blocker can be in a suitable nontoxic pharmacological carrier.

The pharmacological preparation can comprise an ACE inhibitor. The ACE inhibitor can be in a suitable nontoxic pharmacological carrier.

The pharmacological preparation can comprise an angiotensin receptor blocker. The angiotensin receptor blocker can be in a suitable nontoxic pharmacological carrier.

An effective amount for treatment of gingival recession is administered. An amount of calcium channel blocker that is suitable for treatment by topical administration or injection to treat gingival recession is administered.

An effective amount for treatment of gingival recession is administered. An amount of ACE inhibitor that is suitable for treatment by topical administration or injection to treat gingival recession is administered.

An effective amount for treatment of gingival recession is administered. An amount of angiotensin receptor blocker that is suitable for treatment by topical administration or injection to treat gingival recession is administered.

Contact vasodilators (e.g., calcium channel blockers, ACE inhibitors, angiotensin receptor blockers, nitrates, alpha blockers, beta blockers, hydralazine, and/or angiotensin receptor-neprilysin inhibitors) are a new class of pharmaceutical medications that increase blood supply, which produces biological changes.

In the case of treatment or prevention of gingival recession, these changes can include one or more of increasing the blood supply to the tissues, increase the capillary network and blood supply to cartilage, bones, ligaments, and soft tissues.

Calcium channel blockers are a new class of pharmaceutical drugs that disrupt the entry of calcium molecules through the L type voltage operated channels to cardiac muscle and blood vessels cells. The blockage of calcium entry causes the relief of arterial spasm.

Currently there are 70 pharmaceutical patented calcium channel blocker drugs that use this property to treat hypertension, angina pectoris and cardiac arrhythmia. The clinical indication for the therapeutic use of calcium channel blockers was therefore limited, until now, to the field of cardiovascular diseases only.

Calcium channel blockers were extensively studied but their ability to prevent and or to treat gingival recession remained heretofore unknown.

Accordingly, new uses are provided of contact-applied calcium channel blockers for topical application to the gum or by injection into the gum. The new use may be used for the treatment of gingival recession. No trial of topical calcium channel blockers for the prevention or treatment of gingival recession has heretofore been published.

Contact calcium channel blockers are a part of contact-vasodilators, a new class of medication. The use is provided of contact neo-vasodilators such as Nifedipine, a known calcium channel blocker used in the treatment of hypertension, for the treatment of gingival recession.

Nifedipine, Amlodipine, Felodipine, Isradipine, Nicardipine, Nisoldipine and Clevidipine are in a class of dihydropyridines calcium channel blockers. Verapamil and Diltiazem are non-dihydropyridines calcium channel blockers. When applied by contact these are very effective drugs for the treatment of gingival recession.

Inhibitors of angiotensin converting enzyme (ACE) can be employed as vasodilators. Angiotensin II is a chemical produced by the body that primarily circulates in the blood. It causes the muscles surrounding blood vessels to contract, thereby narrowing the vessels. Angiotensin II is formed from angiotensin I in the blood by the enzyme angiotensin converting enzyme (ACE). Angiotensin I in the blood is itself formed from angiotensinogen, a protein produced by the liver and released into the blood. Angiotensin converting enzyme inhibitors (ACE inhibitors) are medications that slow (inhibit) the activity of the enzyme ACE, which decreases the production of angiotensin II. As a result, blood vessels enlarge or dilate. ACE inhibitors include, but are not limited to benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik).

Angiotensin II receptor blockers (ARBs) help relax the blood vessels. Angiotensin II receptor blockers block the action of angiotensin II, allowing blood vessels to dilate. Angiotensin receptor blockers include, but are not limited to: azilsartan (Edarbi), candesartan (Atacand), eprosartan, irbesartan (Avapro), losartan (Cozaar), olmesartan (Benicar), telmisartan (Micardis), and valsartan (Diovan).

Other vasodilators are known in the art. These include, but are not limited to nitrates (nitroglycerin, isosorbide mononitrate and isosorbide dinitrate), Alpha blockers (doxazosin (Cardura), prazosin (Minipress), terazosin), Beta blockers (Acebutolol (Sectral), Atenolol (Tenormin), Bisoprolol fumarate (Zebeta), Carvedilol (Coreg)—Combined alpha/beta blocker, Esmilol (Brevibloc), Labetalol (Trandate, Normodyne)—Combined alpha/beta blocker, Metoprolol tartrate (Lopressor) and metoprolol succinate (Toprol-XL), Nadolol (Corgard), Nebivolol (Bystolic), Penbutolol sulfate (Levatol), Propranolol (Inderal), Sotalol (Betapace), HCTZ and bisoprolol (Ziac) is a beta blocker plus diuretic), Hydralazine, and angiotensin receptor-neprilysin inhibitors (ARNi) (Entresto, sacubitril/valsartan).

### Conditions Amenable to Treatment or Prevention

Compositions and methods are provided for the prevention or treatment of gingival recession, e.g., age related gingival recession.

Application of vasodilators (e.g., calcium channel blockers, ACE inhibitors, angiotensin receptor blockers, nitrates, alpha blockers, beta blockers, hydralazine, and/or angiotensin receptor-neprilysin inhibitors), such as Nifedipine or other calcium channel blockers, which previously may have been used in the treatment of high blood pressure, in a pharmacological composition, in an effective amount, in a contact form, such as, but not limited to an oil, liquid preparation or suspension, or toothpaste, to the gum or for administration by injection into the tissue of the gum, can be employed to treat the symptoms of gingival recession.

Pharmacological compositions of the embodiments include but are not limited to one or more vasodilators (e.g., calcium channel blockers, ACE inhibitors, angiotensin receptor blockers, nitrates, alpha blockers, beta blockers, hydralazine, and/or angiotensin receptor-neprilysin inhibitors) and a suitable non toxic pharmaceutical carrier. The pharmaceutical composition in administered in an amount effective for treating gingival recession, e.g., an amount suitable for treatment by topical application or injection.

Gingival recession, associated symptoms, and treatment thereof, or vasodilators are described in the following references, each of which is incorporated by reference herein in its entirety and each of which is hereby made a part of this specification: Periodontal A A P. Glossary of Periodontal Terms 2001; Holmstrup P, Poulsen A H, Andersen L, et al. Oral infections and systemic diseases. Dent Clin North Am 2003; 47:575. www.who.int/mediacentre/factsheets/fs318/en/ (Accessed on Nov. 5, 2012); Miyazaki H. A global overview of periodontal epidemiology. In: Periodontal needs of developing nations, International Academy of Periodontology, Pack ARC, Newman H N (Eds), Sciences Reviews Limited, Middlesex, N.J. 1996; Marcus S E, Drury T F, Brown L J, Zion G R. Tooth retention and tooth loss in the permanent dentition of adults: United States, 1988-1991. J Dent Res 1996; 75 Spec No:684; Epidemiology of Periodontal diseases. US. Public health service. National center for health statistics. Periodontal diseases in adults, United States. 1960-1962 Washington DC: Government printing Office 1965 PHS publication n1000 series 11 no 12; Epidemiology of Periodontal Diseases 10. Olive R C PERIODONTAL DISEASES IN THE UNITED STATES; Eke P I, Dye B A, Wei L, et al. Prevalence of periodontitis in adults in the United States: 2009 and 2010. J Dent Res 2012; 91:914; Eke P I, Dye B A, Wei L, et al. Prevalence of periodontitis in adults in the United States: 2009 and 2010. J Dent Res 2012; 91:914; Gorman W J prevalence and etiology of gingival recession. J Per odontol 1967 38 [4] 316-22 [.PubMed 5230025]; www.who.int/mediacentre/factsheets/fs318/en/ (Accessed on Nov. 5, 2012); Oliver R C, Brown L J, Löe H. Periodontal diseases in the United States population. J Periodontol 1998; 69:269; Beltrán-Aguilar E D, Barker L K, Canto M T, et al. Surveillance for dental caries, dental sealants, tooth retention, edentulism, and enamel fluorosis—United States, 1988-1994 and 1999-2002. MMWR Surveill Summ 2005; 54:1; Pathogenesis of gingival recession Dental clinics of North A M 2915 OCTOBER 69[4]981-006; Etiology and severity of gingival recessions in an adult population sample in Greece. Dent research journal 2011 spring 8[2] 64-70; Johnson M C, Bozzola J J, Shechmeister I L, Shklair I L. Biochemical study of the relationship of extracellular glucan to adherence and cariogenicity in Streptococcus mutans and an extracellular polysaccharide mutant. J Bacteriol 1977; 129:351; Loesche W. Dental caries and periodontitis: contrasting two infections that have medical implications. Infect Dis Clin North Am 2007; 21:471; Winn D M, Brunelle J A, Selwitz R H, et al. Coronal and root caries in the dentition of adults in the United States, 1988-1991. J Dent Res 1996; 75 Spec No:642; Ronderos M, Michalowicz B. Epidemiology of Periodontal Diseases and Risk Factors. In: Periodontics Medicine, Surgery, and Implants, Mealey B, Genco R, Cohen W (Eds), Elsevier Mosby, St Louis 2004; Fransson C, Mooney J, Kinane D F, Berglundh T. Differences in the inflammatory response in young and old human subjects during the course of experimental gingivitis. J Clin Periodontol 1999; 26:453-460; U.S. Public Health Service, National Institute of Dental Research. Oral Health of United States Adults; National Findings. Bethesda, Md.: National Institute of Dental Research; 1987. NIH publication number 87-2868; Cullinan M P, Westerman B, Hamlet S M, et al. A longitudinal study of interleukin-1 gene polymorphisms and periodontal disease in a general adult population. J Clin Periodontol 2001; 28:1137-1144; Michalowicz B S, Diehl S R, Gunsolley J C, et al. Evidence of a substantial genetic basis for risk of adult periodontitis. J Periodontol 2000; 71:1699-1707; Anerud A, Löe H, Boysen H, Smith M. The natural history of periodontal disease in man; changes in gingival health and oral hygiene before 40 years of age. J Periodontal Res 1979; 14:526-540; Löe H, Anerud A, Boysen H, Morrison E. Natural history of periodontal disease in man. Rapid, moderate and no loss of attachment in Sri Lankan laborers 14 to 46 years of age. J Clin Periodontol 1986; 13:431-445; J Clin Periodontol. 2015 April; 42 Suppl 16:S237-55. doi: 10.1111/jcpe.12330; Ismail A I, Burt B A, Eklund S A. Epidemiologic patterns of smoking and periodontal disease in the United States. J Am Dent Assoc 1983; 106:617-621; Amarasena N, Ekanayaka A N, Herath L, Miyazaki H. Tobacco use and oral hygiene as risk indicators for periodontitis. Community Dent Oral Epidemiol 2002; 30:115-123; Tilakaratne A, Soory M, Ranasinghe A W, Corea S M, Ekanayake S L, de Silva M. Effects of hormonal contraceptives on the periodontium, in a population of rural Sri-Lankan women. J Clin Periodontol 2000; 27:753-757; Preshaw P M, Knutsen M A, Mariotti A. Experimental gingivitis in women using oral contraceptives. J Dent Res 2001; 80:2011-2015; Tonetti M S, D'Aiuto F, Nibali L, et al. Treatment of periodontitis and endothelial function. N Engl J Med 2007; 356:911. Montebugnoli L, Servidio D, Miaton R A, et al. Periodontal health improves systemic inflammatory and haemostatic status in subjects with coronary heart disease. J Clin Periodontol 2005; 32:188; Dietrich T, Jimenez M, Krall Kaye E A, et al. Age-dependent associations between chronic periodontitis/edentulism and risk of coronary heart disease. Circulation 2008; 117:1668; Montebugnoli L, Servidio D, Miaton R A, et al. Periodontal health improves systemic inflammatory and haemostatic status in subjects with coronary heart disease. J Clin Periodontol 2005; 32:188. De Nardin E. The role of inflammatory and immunological mediators in periodontitis and cardiovascular disease. Ann Periodontol 2001; 6:30; Williams, B., Drug discovery in renin-angiotensin system intervention: past and future. Therapeutic Advances in Cardiovascular Disease, 2016 June; 10(3): 118-25; Shi L, Mao C, Xu Z, Zhang L. Angiotensin-converting enzymes and drug discovery in cardiovascular disease. Drug Discovery today, 2010, May 1; 15(9-10):332-41; Godfraind, T., 2017. Discovery and development of calcium channel blockers. Frontiers in pharmacology, 8, p. 286.

Compositions including one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), optionally in combination with conventional therapies, and associated methods for treatment of gingival recession and related symptoms are provided.

Some embodiments relate to a pharmaceutical composition and method of treatment using the pharmaceutical composition, wherein the pharmaceutical composition comprises at least one calcium channel blocker, for example, a calcium channel blocker selected from the group consisting of amlodipine (Norvasc), diltiazem (Cardizem LA, Tiazac), felodipine (Plendil), isradipine (Dynacirc), nifedipine (Adalat, Procardia), nicardipine (Cardene), nimodipine (Nimotop), nisoldipine (Sular), verapamil (Covera-HS, Verelan PM, Calan), verapamil, diltiazem and nicardipine (Cardene IV). Some embodiments relate to a pharmaceutical composition and method of treatment using the pharmaceutical composition, wherein the pharmaceutical composition comprises at least one ACE inhibitors, for example at least one ACE inhibitor selected from the group consisting of benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik). Some embodiments relate to a pharmaceutical composition and method of treatment using the pharmaceutical composition, wherein the pharmaceutical composition comprises at least one angiotensin receptor blocker, for example at least one angiotensin receptor blocker selected from the group consisting of azilsartan (Edarbi), candesartan (Atacand), eprosartan, irbesartan (Avapro), losartan (Cozaar), olmesartan (Benicar), telmisartan (Micardis), and valsartan (Diovan). In certain embodiments, the pharmaceutical composition is in a form suitable for contact administration, e.g., to gum tissue, or by injection administration, e.g., to gum tissue, however other routes of administration are also considered that involve contact of the vasodilator to the tissue to be treated.

The pharmaceutical compositions for treatment of gingival recession can further comprise other pharmaceutically active ingredients. These can include drugs to control pain, for example, nonsteroidal anti-inflammatory drugs such as ibuprofen or naproxen sodium, topical anesthetics such as lidocaine, a drug to fight infections (e.g., antibiotic, antiviral, or antifungal agents). The treatment can be administered in conjunction with other therapies, e.g., the conventional therapies for gingival recession as described elsewhere herein.

The use of topical vasodilators (e.g., calcium channel blockers, ACE inhibitors, angiotensin receptor blockers, nitrates, alpha blockers, beta blockers, hydralazine, and/or angiotensin receptor-neprilysin inhibitors) for treatment of gingival recession is a new class of drugs. The new class may be used for treatment of gingival recession, or to enhance efficacy of conventional gingival recession drugs.

In one method of the vasodilator may be applied directly to the gum to treat or prevent gingival recession.

In another embodiment, the vasodilator may be injected directly into the gum to treat or prevent gingival recession. The vasodilator may be applied even after the gingival recession has been ameliorated to prevent recurrence of gingival recession.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, and phosphates.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, tocopherols, carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more pharmacologically active ingredients (e.g. vasodilators) disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles, and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

It is to be understood that where compounds disclosed herein (e.g., calcium channel blockers, ACE inhibitors, angiotensin receptor blockers, nitrates, alpha blockers, beta blockers, hydralazine, and/or angiotensin receptor-neprilysin inhibitors) have unfilled valencies, then the valencies are to be filled with hydrogens or isotopes thereof, e.g., hydrogen-1 (protium) and hydrogen-2 (deuterium).

It is understood that the compounds described herein (e.g., calcium channel blockers, ACE inhibitors, angiotensin receptor blockers, nitrates, alpha blockers, beta blockers, hydralazine, and/or angiotensin receptor-neprilysin inhibitors) can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein may include crystalline forms (also known as polymorphs, which include the different crystal packing arrangements of the same elemental composition of a compound), amorphous phases, salts, solvates, and hydrates, e.g., of vasodilators. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein (e.g., vasodilators) may exist in unsolvated as well as solvated forms. In general, the solvated forms are considered equivalent to the unsolvated forms for the purposes of the compounds and methods provided herein.

Where a range of values is provided, it is understood that the upper and lower limit, and any intervening value between the upper and lower limit of the range is included.

Any percentages, ratios or other quantities referred to herein are on a weight basis, unless otherwise indicated.

### Pharmaceutical Compositions

The vasodilators (e.g., calcium channel blockers, ACE inhibitors, angiotensin receptor blockers, nitrates, alpha blockers, beta blockers, hydralazine, and/or angiotensin receptor-neprilysin inhibitors) can be prepared by any suitable method known to those in the art. For representative methods, see, for example, Francis A. Carey et al., Advanced Organic Chemistry: Part B: Reaction and Synthesis (5^{th} Ed. 2005).

Formulations including a vasodilator (e.g., a calcium channel blocker, ACE inhibitor and/or angiotensin receptor blocker) and at least one excipient are provided. It is generally preferred to administer the compounds of the embodiments in topical formulations, e.g., a gel or a toothpaste; however, other routes of administration, such as injection, are also contemplated.

The pharmaceutical compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The pharmaceutical compositions disclosed herein may be manufactured by a process that is itself known, e.g., by means of conventional mixing, dissolving, granulating, dragee-making, levigating, emulsifying, encapsulating, entrapping, tableting, or extracting processes. Many of the vasodilator (e.g., a calcium channel blocker, ACE inhibitor and/or angiotensin receptor blocker) used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intra-articular, intra-tissue, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions described herein are suitable for use in treatment or prevention of gingival recession or associated symptoms. The compositions are suitable for use in any patient where treatment or prevention of gingival recession is desirable.

The vasodilator (e.g., a calcium channel blocker, ACE inhibitor and/or angiotensin receptor blocker) can be employed in various types of formulations. Topical formulations including one or more vasodilators in combination with at least one excipient are provided. Excipients can include a nonaqueous or aqueous carrier, and one or more agents selected from moisturizing agents, pH adjusting agents, deodorants, fragrances, chelating agents, preservatives, emulsifiers, thickeners, solubilizing agents, penetration enhancers, anti-irritants, colorants, flavorants, surfactants, beneficial agents, pharmaceutical agents, and other components as known in the art for use in connection with topical formulations for application to gum tissue or the oral cavity. The formulation can be provided as an aqueous formulation, or in an anhydrous formulation which may prevent water-based irritant contact dermatitis or stinging sensation upon application. In another embodiment, the composition is formulated such that preservatives need not be employed (e.g., a preservative-free formulation) so as to avoid skin irritation associated with certain preservatives.

To facilitate application, the composition may be provided as an ointment, an oil, a lotion, a paste, a powder, a gel, toothpaste, or a cream. The composition may also include additional ingredients such as a protective agent, an emollient, a humectant, an antibiotic agent, an antifungal agent, an antiviral agent, an antiprotozoal agent, an anesthetic agent, a steroidal anti-inflammatory agent, a non-steroidal anti-inflammatory agent, an antipruritic agent, an antioxidant agent, an anti-histamine agent, a vitamin or vitamin complex, a hormone, an anti-skin atrophy agent, and combinations thereof. In a further embodiment, the composition may avoid animal or cellular-based materials to avoid irritation. The composition can be applied directly to the gum or by injection into gum tissue.

Methods of using vasodilator formulations are provided. The compositions may be applied topically in the region of gingival recession, but may also be applied to gum tissues by injection.

Some embodiments include administering vasodilator (e.g., a calcium channel blocker, ACE inhibitor and/or angiotensin receptor blocker) compositions provided herein in topical formulations; however, other routes of administration are also contemplated (e.g., intra-tissue injection or the like). Contemplated routes of administration include but are not limited to topical and injection. Suitable liquid forms include suspensions, emulsions, solutions, and the like. Unit dosage forms can also be provided, e.g., individual packets with a premeasured amount of the formulation, configured for administration to the tissue on a predetermined schedule (e.g., daily, weekly, etc.). Unit dosage forms configured for administration twice a day can be employed; however, in certain embodiments it can be desirable to configure the unit dosage form for administration once a day, four times a day, or more, or once every other day, every three days, weekly, or less, or on an as-needed basis.

In some embodiments, the topical and injectable formulations typically comprise from about 0.001 wt. % or less to about 50 wt. % or more of active ingredient, such as the vasodilator (e.g., a calcium channel blocker, ACE inhibitor and/or angiotensin receptor blocker), preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Compositions and formulations for topical administration to the tissue of the gum can include gels, drops, sprays, liquids, and aerosols. Conventional pharmaceutical carriers, aqueous or oily bases, pastes, thickeners and the like may be employed.

A topical formulation can be provided in a form of a carrier containing the vasodilator, e.g., 50 ppm or less to 1000, 5000, 10000, 50000, 100000, 500000 ppm or more of the vasodilator. The topical formulation can contain from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 10 wt. % or 20 wt. % of the vasodilator. The amount of vasodilator in the base can be adjusted up or down.

Liquids and gels containing the vasodilator, optionally with other components as described herein, can be prepared using techniques as are known in the art for preparing topical compositions. See, e.g., Handbook of Cosmetic Science and Technology, Fourth Edition, edited by André O. Barel, Marc Paye, Howard I. Maibach, CRC Press, 2014, the contents of which is hereby incorporated by reference in its entirety. Various formulations are possible.

For liquid formulations (e.g., gel or lotion forms), a silicone, e.g., a cyclosiloxane or linear silicone (e.g., silicone elastomer), can be employed as a carrier. One type of suitable carrier is a dimethicone crosspolymer gel, e.g., dimethicone crosspolymer in cyclopentasiloxane. Other suitable dimethicone crosspolymers include cyclopentasiloxane, dimethicone/vinyldimethicone crosspolymer; dimethicone, dimethicone/vinyl dimethicone crosspolymer; and isodecane dimethicone/vinyl dimethicone crosspolymer.

Typically, the carrier is present in an amount of from about 80 wt. % to about 95 wt. %, or 82 wt. % to 92 wt. %, e.g., in a topical formulation for application to gum.

Penetration enhancers can be employed to enhance penetration of the vasodilator into tissue. Typical amounts when employed in topical formulations are from 1% by weight to 4% by weight. Typical amounts for anti-irritation agents when employed in topical formulations are from 1% by weight to 4% by weight. Typical amounts for anti-inflammatory agents when employed in topical formulations are from 1% by weight to 4% by weight. Typical amounts for anti-inflammatory agents when employed in topical formulations are from 0.1% by weight to 2% by weight.

In some embodiments, the vasodilator can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration to the tissue of the gum comprise the vasodilator as described herein and a vehicle acceptable for contact with gum tissue. The vehicle may be aqueous or nonaqueous. The vehicle used in the topical composition may be in the form of a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the vasodilator into the gum tissue.

The pharmaceutical excipients used in the topical preparations of the vasodilator compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for hydrophobic topical formulations include mineral oils, vegetable oils, and silicone oils. If desired, the vasodilator compositions as described herein may be dissolved or dispersed in a hydrophobic oil phase, and the oil phase may then be emulsified in an aqueous phase comprising water, alone or in combination with lower alcohols, glycerin, and/or glycols. In certain embodiments water is present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the formulations as described herein can be advantageously employed where it is desirable to minimize osmotic shock.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carragenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

Lecithin and other phospholipids may be used to prepare liposomes containing the vasodilators as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the vasodilators as described herein.

The topical formulation may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The vasodilator compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When the vasodilator formulations of the embodiments are administered by injection, it is preferably in the form of a pyrogen-free, parenterally acceptable aqueous solution or oleaginous suspension, emulsion or solution. Suspensions can be formulated according to methods well known in the art using suitable dispersing or wetting agents and suspending agents. The preparation of acceptable aqueous or nonaqueous solutions with suitable properties, e.g., pH, isotonicity, stability, and the like, is within the skill in the art. For example, an isotonic vehicle such as 1,3-butanediol, water, isotonic sodium chloride solution, Ringer's solution, dextrose solution, dextrose and sodium chloride solution, lactated Ringer's solution, or other vehicles as are known in the art can be employed, or a fixed oil can be employed conventionally as a solvent or suspending medium, e.g., synthetic mono or diglycerides, fatty acids, or the like. The vasodilator formulations can also contain stabilizers, preservatives, buffers, antioxidants, or other additives known to those of skill in the art.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

### Kits For Administration of Compositions

Some embodiments of the methods and compositions provided herein include kits comprising vasodilators provided herein. In some embodiments, kits can be provided to an administering physician, other health care professional, a patient, or a caregiver. In some embodiments, a kit comprises a container which contains the vasodilator(s) in a suitable topical formulation, and instructions for administering the composition to a subject. The kit can optionally also contain one or more additional therapeutic or other agents. For example, a kit containing a vasodilator blocker in topical form can be provided along with other agents such as topical antibiotics or topical anesthetics. The kit may contain the vasodilator in bulk form, or can contain separate doses of the vasodilator for serial or sequential administration. The kit can optionally contain one or more diagnostic tools, administration tools, and/or instructions for use, e.g., syringes for injection. The kit can contain suitable delivery devices, such as, sticks, syringes, pump dispensers, tubes, wands, single dose packets, and the like, along with instructions for administering the vasodilator compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

The topical formulation for administration to tissue of the gum, in addition to the vasodilator, can contain other ingredients.

While topical administration of the vasodilator disclosed herein can advantageously be employed, in certain embodiments other routes of administration are also contemplated, such as injection.

The vasodilator compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The vasodilator compositions disclosed herein may be manufactured into administrable forms by a process that is itself known, e.g., by means of conventional mixing, dissolving, granulating, dragee-making, levigating, emulsifying, encapsulating, entrapping, tableting, or extracting processes.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal, intra-articular, intra-tis sue, and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the vasodilator may be combined as the active ingredient in intimate admixture with a pharmaceutical carrier according to conventional pharmaceutical compounding techniques. The excipients are preferably minimized so as to ensure administration of an appropriate amount of vasodilator in a compact format. The carrier can take a wide variety of forms depending on the form of preparation desired for administration. Thus, the vasodilator compositions provided herein can be presented as discrete units suitable for administration each containing a predetermined amount of the active ingredient. Further, the vasodilator compositions can be presented as an oil, as a solution, as a suspension in an aqueous liquid, as a non-aqueous liquid, as an oil-in-water emulsion, or as a water-in-oil liquid emulsion, or toothpaste, similar to the topical formulations described elsewhere herein, but using components suitable for human contact or consumption. In addition to the common dosage forms set out above, the vasodilator compositions provided herein can also be administered by controlled release and/or delivery devices. The vasodilator compositions can be prepared by any of the methods of pharmacy. In general, such methods include a step of bringing into association the active ingredient with the carrier that constitutes one or more necessary ingredients. In general, the vasodilator compositions are prepared by uniformly and intimately admixing the vasodilator ingredient(s) with liquid carriers or finely divided solid carriers or both. The product can then be conveniently shaped into the desired presentation.

A vasodilator formulation may also be administered in a local manner, for example, via injection of the vasodilator composition directly into a target area, e.g., in a depot or sustained release formulation in the gum or region of the gum. Furthermore, a targeted drug delivery system for the vasodilator may be used, for example, in a liposome coated with a tissue specific antibody.

The vasodilator compositions may contain the vasodilator in an amount effective for the desired therapeutic effect. In some embodiments, the vasodilator compositions are in a unit dosage form and comprise from about 0.1 mg or less to about 5000 mg or more of vasodilator per unit dosage form. In further embodiments, the vasodilator compositions comprise from about 1 to about 500 mg per unit dosage form or from about 500 to 5000 mg per unit dosage form of vasodilator. Such amounts can be selected depending upon the vasodilator employed. Such dosage forms may be solid, semisolid, liquid, an emulsion, or adapted for delivery via aerosol or the like.

The carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

Vasodilator compositions provided herein can be prepared as solutions or suspensions of the vasodilator in water or nonaqueous liquids. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Vasodilator compositions provided herein suitable for injectable use include sterile aqueous solutions or dispersions. Furthermore, the vasodilator compositions can be in the form of sterile powders for the extemporaneous preparation of such sterile injectable solutions or dispersions. The vasodilator compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the vasodilator formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood or other bodily fluids of the intended recipient. Vasodilator compositions can also be prepared in powder or liquid concentrate form for dilution.

Contemplated herein are vasodilator compositions including one or more vasodilators as described herein in combination with at least one additional active agent, e.g., an antibiotic. The vasodilator and the at least one additional active agent(s) may be present in a single formulation or in multiple formulations provided together, or may be unformulated. In some embodiments, the vasodilator can be administered with one or more additional agents together in a single composition. For example, the vasodilator can be administered in one composition, and at least one of the additional agents can be administered in a second composition. In a further embodiment, the vasodilator and the at least one additional active agent(s) are co-packaged in a kit. For example, a drug manufacturer, a drug reseller, a physician, a compounding shop, or a pharmacist can provide a kit comprising the vasodilator in combination with another product or component for delivery to a patient. Such additional components can include anti-infective agents, anti-inflammatory agents, anesthetics, or the like.

Some embodiments described herein relate to compositions of vasodilator, which can include a therapeutically effective amount of the vasodilator described herein and a pharmaceutically acceptable carrier, diluent, excipient or combination thereof. The vasodilator composition can include the vasodilator in an amount for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥≥90%, ≥95%, or ≥98% of the composition.

### EXAMPLES

### Example 1

A patient is diagnosed with gingival recession throughout the mouth. A composition comprising the calcium channel blocker nifedipine is directly applied to the tissue of the gum on one side of the mouth while the other side is left untreated. The treated side is observed to have reduced symptoms of gingival recession than the untreated side.

### Example 2

A patient is diagnosed with gingival recession throughout the mouth. A composition comprising the ACE inhibitor enalapril is directly applied to the tissue of the gum on one side of the mouth while the other side is left untreated. The treated side is observed to have reduced symptoms of gingival recession than the untreated side.

### Example 3

A patient is diagnosed with gingival recession throughout the mouth. A composition comprising the angiotensin receptor blocker losartan is directly applied to the tissue of the gum on one side of the mouth while the other side is left untreated. The treated side is observed to have reduced symptoms of gingival recession than the untreated side.

### Example 4

A patient is diagnosed with gingival recession throughout the mouth. A composition comprising the calcium channel blocker nifedipine is administered by injection into the tissue of the gum on one side of the mouth while the other side is left untreated. The treated side is observed to have reduced symptoms of gingival recession than the untreated side.

### Example 5

A patient is diagnosed with gingival recession throughout the mouth. A composition comprising the ACE inhibitor enalapril is administered by injection into the tissue of the gum on one side of the mouth while the other side is left untreated. The treated side is observed to have reduced symptoms of gingival recession than the untreated side.

### Example 6

A patient is diagnosed with gingival recession throughout the mouth. A composition comprising the angiotensin receptor blocker losartan is administered by injection into the tissue of the gum on one side of the mouth while the other side is left untreated. A treated side is observed to have reduced symptoms of gingival recession than the untreated side.

### Example 7

A patient is diagnosed with gingival recession throughout the mouth and successfully treated for the condition. A composition comprising the calcium channel blocker nifedipine is directly applied to the tissue of the gum on one side of the mouth while the other side is left untreated. After a period of time, the treated side is observed to be free of symptoms of gingival recession, while the untreated side again exhibits symptoms of gingival recession.

### Example 8

A patient is diagnosed with gingival recession throughout the mouth and successfully treated for the condition. A composition comprising the ACE inhibitor enalapril is directly applied to the tissue of the gum on one side of the mouth while the other side is left untreated. After a period of time, the treated side is observed to be free of symptoms of gingival recession, while the untreated side again exhibits symptoms of gingival recession.

### Example 9

A patient is diagnosed with gingival recession throughout the mouth and successfully treated for the condition. A composition comprising the angiotensin receptor blocker losartan is directly applied to the tissue of the gum on one side of the mouth while the other side is left untreated. After a period of time, the treated side is observed to be free of symptoms of gingival recession, while the untreated side again exhibits symptoms of gingival recession.

### Example 10

A patient is diagnosed with gingival recession throughout the mouth and successfully treated for the condition. A composition comprising the calcium channel blocker nifedipine is administered by injection into the tissue of the gum on one side of the mouth while the other side is left untreated. After a period of time, the treated side is observed to be free of symptoms of gingival recession, while the untreated side again exhibits symptoms of gingival recession.

### Example 11

A patient is diagnosed with gingival recession throughout the mouth and successfully treated for the condition. A composition comprising the ACE inhibitor enalapril is administered by injection into the tissue of the gum on one side of the mouth while the other side is left untreated. After a period of time, the treated side is observed to be free of symptoms of gingival recession, while the untreated side again exhibits symptoms of gingival recession.

### Example 12

A patient is diagnosed with gingival recession throughout the mouth and successfully treated for the condition. A composition comprising the angiotensin receptor blocker losartan is administered by injection into the tissue of the gum on one side of the mouth while the other side is left untreated. After a period of time, the treated side is observed to be free of symptoms of gingival recession, while the untreated side again exhibits symptoms of gingival recession.

### Exemplary Pharmaceutical Compositions and Methods

Pharmaceutical Composition 1: A pharmaceutical composition for the treatment or prophylaxis of gingival recession e, comprising: at least one vasodilator; and at least one pharmaceutical excipient.

Pharmaceutical Composition 2: Pharmaceutical Composition 1, for the treatment of gingival recession.

Pharmaceutical Composition 3: Pharmaceutical Composition 1, for the prevention of recurrence of gingival recession in a patient previously diagnosed and successfully treated for gingival recession.

Pharmaceutical Composition 4: Any one of Pharmaceutical Compositions 1 through 3, in a form adapted for topical administration or injection to gum tissue.

Pharmaceutical Composition 5: Pharmaceutical Composition 4, wherein the form is selected from the group consisting of an oil, a liquid, a suspension, and a paste for topical application on the gum.

Pharmaceutical Composition 6: Any One of Pharmaceutical Compositions 1 through 3, formulated as a liquid or a suspension of the at least one vasodilator, wherein the vasodilator is a contact vasodilator.

Pharmaceutical Composition 7: Any One of Pharmaceutical Compositions 1 through 6, wherein the vasodilator is a calcium channel blocker.

Pharmaceutical Composition 8: Pharmaceutical Composition 7, wherein the at least one calcium channel blocker is a dihydropyridine selected from the group consisting of nifedipine, isradipine, felodipine, amlodipine, nicardipine, and clevidipine.

Pharmaceutical Composition 9: Pharmaceutical Composition 7, wherein the at least one calcium channel blocker is a non dihydropyridine selected from the group consisting of verapamil and diltiazem.

Pharmaceutical Composition 10: Any One of Pharmaceutical Compositions 1 through 6, wherein the vasodilator is an ACE inhibitor.

Pharmaceutical Composition 11: Pharmaceutical Composition 10, wherein the ACE inhibitor is selected from the group consisting of benazepril, captopril, enalapril, fosinopril, lisinopril, moexipril, perindopril, quinapril, ramipril, and trandolapril.

Pharmaceutical Composition 12: Any One of Pharmaceutical Compositions 1 through 6, wherein the vasodilator is an angiotensin receptor blocker.

Pharmaceutical Composition 13: Pharmaceutical Composition 12, wherein the angiotensin receptor blocker is selected from the group consisting of azilsartan, candesartan, eprosartan, irbesartan, losartan, olmesartan, telmisartan, and valsartan.

Pharmaceutical Composition 14: Any One of Pharmaceutical Compositions 1 through 6, wherein the vasodilator is a nitrate.

Pharmaceutical Composition 15: Pharmaceutical Composition 14, wherein the nitrate is selected from the group consisting of nitroglycerin, isosorbide mononitrate and isosorbide dinitrate.

Pharmaceutical Composition 16: Any One of Pharmaceutical Compositions 1 through 6, wherein the vasodilator is an alpha blocker.

Pharmaceutical Composition 17: Pharmaceutical Composition 16, wherein the alpha blocker is selected from the group consisting of doxazosin, prazosin, and terazosin.

Pharmaceutical Composition 18: Any One of Pharmaceutical Compositions 1 through 6, wherein the vasodilator is a beta blocker.

Pharmaceutical Composition 19: Pharmaceutical Composition 18, wherein the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

Pharmaceutical Composition 20: Any One of Pharmaceutical Compositions 1 through 6, wherein the vasodilator is hydralazine.

Pharmaceutical Composition 21: Any One of Pharmaceutical Compositions 1 through 6, wherein the vasodilator is an angiotensin receptor-neprilysin inhibitor.

Pharmaceutical Composition 22: Pharmaceutical Composition 21, wherein the angiotensin receptor-neprilysin inhibitor is sacubitril/valsartan.

Pharmaceutical Composition 23: Any one of Pharmaceutical Compositions 1 through 22, wherein the concentration of the vasodilator is about 0.0001 mg per ml to 1000 mg per ml, optionally 1 mg per ml to 10 mg per ml, optionally 1 mg per ml to 1000 mg per ml, optionally 5 mg per ml to 10 mg per ml, optionally 10 mg per ml, optionally 20 mg per ml, optionally 30 mg per ml, optionally 60 mg per ml, optionally 90 mg per ml, optionally 120 mg per ml, optionally 180 mg per ml, optionally 240 mg per ml, optionally more than 10 mg per ml.

Pharmaceutical Composition 24: Any One of Pharmaceutical Compositions 1 through 2, wherein the concentration of the vasodilator is from about 0.0001% by weight to about 20% by weight, optionally about 0.01% by weight, optionally about 0.1% by weight, optionally about 1% by weight, optionally about 10% by weight, optionally about 20% by weight.

Method 25: A method for the treatment or prophylaxis of gingival recession in a patient in need thereof, comprising: administering an effective amount of the pharmaceutical composition according to any one of Pharmaceutical Compositions 1 through 24 to a patient in need thereof.

Method 26: Method 25, for the treatment of gingival recession.

Method 27: Method 25, for the prevention of recurrence of gingival recession in a patient previously diagnosed and successfully treated for gingival recession.

Method 28: Method 25, wherein the composition is administered once a day, optionally two or more times a day, optionally once a week, optionally two or more times a week, optionally once a month, optionally two or more times a month, optionally a plurality of times a year.

Any of the features the above referenced pharmaceutical compositions, uses, and methods is applicable to any other pharmaceutical composition, use, or method identified herein. Moreover, any of the features of the above referenced pharmaceutical compositions, uses, and methods is independently combinable, partly or wholly, with other embodiments of the pharmaceutical compositions, uses, and methods described herein in any way, e.g., one, two, or three or more features may be combinable in whole or in part. Further, any of the features of the pharmaceutical compositions, uses, and methods described above may be made optional to other pharmaceutical compositions, uses, and methods described herein. Any aspect or embodiment of a method or use described herein can be performed using a composition, e.g., a pharmaceutical composition and/or a compound as described herein, and any aspect or embodiment of a composition, e.g., a pharmaceutical composition and/or a compound described herein, can be used or adapted to perform a method or use as described herein.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more' to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A pharmaceutical composition for the treatment or prophylaxis of gingival recession, comprising:
at least one vasodilator; and
at least one pharmaceutical excipient.

2. The pharmaceutical composition of claim 1, wherein the pharmaceutical composition is in a form adapted for topical administration or injection to gum tissue.

3. The pharmaceutical composition of claim 2, wherein the form is selected from the group consisting of an oil, a liquid, a suspension, and a paste for topical application on the gum.

4. The pharmaceutical composition of claim 1, wherein the pharmaceutical composition is formulated as a liquid or a suspension, wherein the vasodilator is a contact vasodilator.

5. The pharmaceutical composition of claim 1, wherein the vasodilator is a calcium channel blocker selected from a dihydropyridine selected from the group consisting of nifedipine, isradipine, felodipine, amlodipine, nicardipine, and clevidipine or a non dihydropyridine selected from the group consisting of verapamil and diltiazem.

6. The pharmaceutical composition of claim 1, wherein the vasodilator is an ACE inhibitor selected from the group consisting of benazepril, captopril, enalapril, fosinopril, lisinopril, moexipril, perindopril, quinapril, ramipril, and trandolapril.

7. The pharmaceutical composition of claim 1, wherein the vasodilator is an angiotensin receptor blocker selected from the group consisting of azilsartan, candesartan, eprosartan, irbesartan, losartan, olmesartan, telmisartan, and valsartan.

8. The pharmaceutical composition claim 1, wherein the vasodilator is a nitrate selected from the group consisting of nitroglycerin, isosorbide mononitrate and isosorbide dinitrate.

9. The pharmaceutical composition of claim 1, wherein the vasodilator is an alpha blocker selected from the group consisting of doxazosin, prazosin, and terazosin.

10. The pharmaceutical composition of claim 1, wherein the vasodilator is a beta blocker.

11. The pharmaceutical composition of claim 10, wherein the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

12. The pharmaceutical composition of claim 1, wherein the vasodilator is hydralazine.

13. The pharmaceutical composition of claim 1, wherein the vasodilator is an angiotensin receptor-neprilysin inhibitor.

14. The pharmaceutical composition of claim 14, wherein the angiotensin receptor-neprilysin inhibitor is sacubitril/valsartan.

15. The pharmaceutical composition of claim 1, wherein the concentration of the vasodilator is about 0.0001 mg per ml to 1000 mg per ml.

16. The pharmaceutical composition of claim 1, wherein the concentration of the vasodilator is from about 0.0001% by weight to about 20% by weight.

17. A method for the treatment or prophylaxis of gingival recession in a patient in need thereof, comprising:
administering an effective amount of the pharmaceutical composition according to claim 1 to a patient in need thereof.

18. The method of claim 17, for the treatment of gingival recession.

19. The method of claim 17, for the prevention of recurrence of gingival recession in a patient previously diagnosed and successfully treated for gingival recession.

20. The method of claim 17, wherein the composition is administered once a day, optionally two or more times a day, optionally once a week, optionally two or more times a week, optionally once a month, optionally two or more times a month, optionally a plurality of times a year.
